(19) United States
(12) Patent Application Publication
(10) Pub. No.: US 2007/0184004 A1
(43) Pub. Date: Aug. 9, 2007

(54) Shea butter dimethicone copolyols

(76) Inventors: Steven Rogers, Yardley, PA (US); Anthony O'Lenick JR., Dacula, GA (US)

Correspondence Address: Louis C. Paul & Associates, PLLC, 9th floor, 730 Fifth Avenue, New York, NY, 10019, US

(21) Appl. No.: 11/347,271

(22) Filed: Feb. 6, 2006

(51) Int. Cl. A61K 8/89; C07F 7/02

(52) U.S. Cl. 424/70.12; 554/77

(57) ABSTRACT

Novel silicone compounds prepared by the reaction of a dimethicone copolyol with shea butter, preferably mild-processed shea butter (MPSB). These compounds are useful as cosmetic and personal care ingredients, allowing for the delivery of highly desirable active ingredients present in shea butter, including natural antioxidants, in compounds are substantive to the skin and hair.

### CROSS-REFERENCE TO RELATED APPLICATIONS

Not applicable.

### STATEMENT REGARDING FEDERALLY-SPONSORED RESEARCH OR DEVELOPMENT

Not applicable.

### FIELD OF THE INVENTION

The present invention relates to novel silicone compounds prepared by the reaction of a dimethicone copolyol with shea butter, preferably mild-processed shea butter. These compounds are useful as cosmetic and personal care ingredients, allowing for the delivery of highly desirable active ingredients present in shea butter, including natural antioxidants, in compounds are substantive to the skin and hair.

### BACKGROUND OF THE INVENTION

Shea Butter is a butter extracted from the kernel of Butrospermum parkii. This plant, also referred to as Vitellaria paradoxa, is native to Africa. The term butter describes a material that is a solid at room temperature, but melts at about 40° C. Chemically, the butter is a triglyceride conforming to the following structure

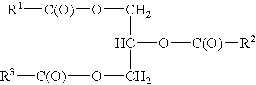

wherein R¹, R² and R³ each have one of the following compositions:

| R Group | Common Name | Range (%) | Typical (%) |
|---|---|---|---|
| C₁₁H₂₃ | Lauryl | 0.1-2.0 | 0.2 |
| C₁₃H₂₇ | Myristyl | 0.5-2.0 | 1.0 |
| C₁₅H₃₁ | Cetyl | 2.0-6.0 | 4.0 |
| C₁₇H₃₅ | Stearyl | 25.0-50.0 | 35.0 |
| C₁₇H₃₃ | Oleyl | 40.0-60.0 | 59.0 |
| C₁₇H₃₁ | Linoleyl | 0.5-1.0 | 0.8 |

The average composition of R² is different than R¹ and R³, the latter two being similar. The R² moiety contains predominantly the unsaturated C₁₈ group (oleyl) while R¹ and R³ contain predominantly the saturated C₁₈ group (stearyl). Differences between internal (R²) and terminal (R¹, R³) substitution are seen in natural products but not in synthetic molecules produced in the laboratory.

The high levels of stearyl and oleyl groups in shea butter and its dimethicone copolyol derivatives make them of particular interest in the personal care industry. While other raw materials used in personal care products have these species, the compounds of the present invention have significantly high concentrations of unsaponifiables, which posses highly desired antioxidant, ultra-violet radiation protection, and free-radical scavenging properties. MPSB of the present invention typically contain from about 5% to about 15% by weight of unsaponifiables. In contrast, other butters commonly used in personal care products have less than 2% unsaponifiables. For example, coca butter (from Theobroma cacao) averages 0.4% unsaponifiables and Illipe butter (from Shorea stenoptera) averages 1.1%.

As described in greater detail below, the novel dimethicone copolyols of the present invention are produced by reacting shea butter and dimethicone copolyol (“DMC”). Preferably, the shea butter is MPSB and is reacted with the DMC under specific mild processing conditions. By “mild processed” is meant processes that do not remove or otherwise diminish the amount or potency of active ingredients, particularly highly desired unsaponifiables. In the present invention, mild processing is employed both at the time of harvesting and initial extraction and during subsequent preparation of derivatives. These mild processes result in materials containing unexpectedly high amounts unsaponifiables, notably antioxidants.

Dimethicone copolyols are silicone-based nonionic surfactants that are widely-used in the cosmetics and personal care industries. With pendant water-soluble polyoxyalkylene groups on a water-insoluble silicone backbone, dimethicone copolyol compounds have many uses, including as formulation aids in creating water-in-silicone and silicone-in water emulsions. Numerous variations on dimethicone copolyols (i.e., in terms of molecular weights, amount of silicone, number of polyoxyalkylene groups) are commercially available.

Dimethicone copolyol esters, prepared by an esterification reaction of dimethicone copolyol, are known in the art. The addition of a fatty group creates a molecule that contains water-, silicone- and oil-soluble groups. Dimethicone copolyol esters made from naturally-derived triglyceride materials are commercially available, and include dimethicone copolyol meadowfoamate, which is used in the Intensive Blends line of hair care products sold by Clairol. See also, A. Wohlman, “A Meadowfoam Seed Oil Derivative and Its Activity on Human Hair” Cosmetics & Toiletries, Vol. 112, pages 83-87 (1997). U.S. Pat. No. 6,630,187 discloses silicone esters of raspberry seed oil. The silicone esters described in the '187 patent do not possess the unsaponifiable fractions, and with them antioxidant properties, of the compounds of the present invention.

### SUMMARY OF THE INVENTION

The compounds of the present invention are shea butter dimethicone copolyols produced by reacting shea butter with dimethicone copolyol. Preferably, the shea butter is mild-processed and is reacted with the DMC under mild conditions. The novel dimethicone copolyols of the present invention are rich in unsaponifiables, including antioxidants.

### DETAILED DESCRIPTION OF THE INVENTION

The present invention relates to a novel class of silicones, namely dimethicone copolyols, made by reacting shea butter with dimethicone copolyol, preferably by reacting mild-processed shea butter under specific mild-processing conditions. The invention also relates to a process for using these compositions in personal care applications. Preferably, mild processing is employed both at the time of harvesting and initial extraction and during subsequent preparation of alkoxylate derivatives. In so doing, materials containing unexpectedly high amounts of active ingredients, particularly highly desired unsaponifiables, are produced.

Shea butter dimethicone copolyols of the present invention conform to the structure: 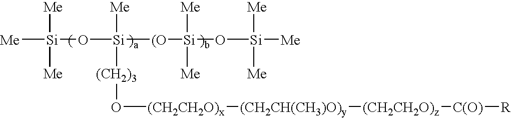
wherein

(i) Me is methyl;

(ii) a is an integer ranging from 1 to 20;

(iii) b is an integer ranging from 0 to 200;

(iv) x, y and z are independently integers ranging from 0 to 20;

(v) R is derived from shea butter and comprises
from about 0.1 to about 2.0% by weight. C₁₁H₂₃;
from about 0.5 to about 2.0% by weight C₁₃H₂₇;
from about 2.0 to about 6.0% by weight C₁₅H₃₁;
from about 25 to about 50% by weight C₁₇H₃₅;
from about 40.0 to about 60.0% by weight C₁₇H₃₃

Shea Butter

Shea butter can be prepared by standard extraction techniques known to those of skill in the art. For example, U.S. Pat. No. 6,552,208, the disclosure of which is incorporated herein by reference, describes several methods for processing shea butter. Suitable extraction vehicles may include, but are not limited to, ethanol, methanol, ethyl acetate, acetone, chloroform and water, or any other solvent and water.

In a preferred aspect of the present invention, shea butter is mild-processed; it is extracted using a hydrocarbon-free solvent system and its dimethicone copolyol derivatives are made under mild processing conditions. At the time of harvesting and initial extraction ground-up kernels are boiled in water under mild conditions as described in the examples below. The oil phase is then separated from the water phase by decanting. This process provides a yellow, solid wax rich in unsaponifiables. By wax is meant a material obtained by boiling in water under ambient conditions, decanted and filtered.

The mild processing of the present invention may be contrasted with separation using solvents and high temperature treatment with high pressure steam. While the latter processes result in what some may describe as a “more pure” triglyceride, unsaponifiables, and the benefits derived therefrom, are lost. Vacuum distillation which strips off the desirable components is also to be avoided in processing MPSB of the present invention. By processing shea butter under mild conditions, materials comprising from about 5% to about 15% by weight of unsaponifiables can be produced.

Sterols comprise about 20% of the unsaponifiables. More particularly, the sterols comprise: cholesterol (from about 1% to about 3%); alpha-spinasterol (from about 1% to about 4%); delta-7-stigmasterol (from about 40% to about 44%); delta-7-avenasterol (from about 38% to about 41%). The remaining constituents of the unsaponifiables (about 80%) include other highly desirable active compounds including tocopherol, karitin, cinamic acid esters, alpha and beta amyrin and phenolics.

Phenolic compounds are natural products composed of one or more aromatic benzene rings with one or more hydroxyl group. They are a class of natural products that possess antioxidant and free radical scavenging properties. Among the phenolics in the unsaponifiables of mild-processed shea butter include gallic acid, gallocatchin, catechin, epigallocatechin gallate, epicatechin, gallocatechin gallate, gallocatechin gallate and quercetin.

Dimethicone copolyols are items of commerce made by hydrosilylation of a polymer containing a silanic hydrogen moiety. They are available from Siltech, LLC (Dacula, Ga.) and conform to the following formula:

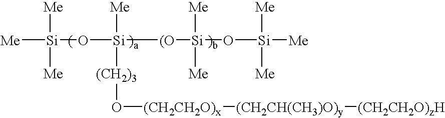

Illustrative dimethicone copolyols suitable for use in the present invention are as follows:

| Example | a | b | x | y | z |
|---|---|---|---|---|---|
| 1 | 1 | 0 | 0 | 0 | 5 |
| 2 | 4 | 0 | 0 | 0 | 5 |
| 3 | 4 | 8 | 5 | 4 | 5 |
| 4 | 4 | 10 | 20 | 20 | 20 |
| 5 | 4 | 100 | 20 | 0 | 20 |
| 6 | 4 | 20 | 5 | 10 | 20 |
| 7 | 10 | 150 | 10 | 15 | 10 |
| 8 | 10 | 200 | 20 | 5 | 20 |
| 9 | 15 | 10 | 0 | 10 | 20 |
| 10 | 20 | 1 | 10 | 10 | 10 |

One aspect of the present invention is a shea butter dimethicone copolyol made by a transesterification reaction, in which one ester is made by using another ester as a raw material. Suitable catalysts for a transesterification reaction are methane sulfonic acid, tin, compound titinate compounds. In a preferred embodiment, the transesterification is conducted at a temperature of from about 150° C. to about 220° C. in the presence of a tin catalyst. A particularly preferred tin catalyst is dilauryl tin oxide.

Another aspect of the present invention is a process for delivering antioxidants to the skin and hair by applying thereto a finished topical product comprising an effective concentration of a shea butter dimethicone copolyol that is made by a transesterification reaction forms to the structure: 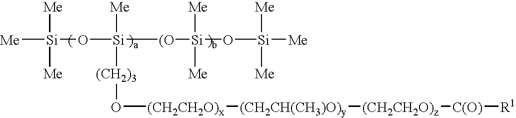
wherein

(i) Me is methyl;

(ii) a is an integer ranging from 1 to 20;

(iii) b is an integer ranging from 0 to 200; (iv) x, y and z are independently integers ranging from 0 to 20;

(v) R¹ is derived from shea butter and comprises
from about 0.1 to about 2.0% by weight C₁₁H₂₃;
from about 0.5 to about 2.0% by weight C₁₃H₂₇;
from about 2.0 to about 6.0% by weight C₁₅H₃₁;
from about 25 to about 50% by weight C₁₇H₃₅;
from about 40.0 to about 60.0% by weight C₁₇H₃₃

By “finished topical product” is meant a cream, lotion, gel, foam, ointment, paste, emulsion, suspension, dispersion, solution, or similar topically-applied carrier or delivery system known to those of skill in the art.

The following examples are further illustrative of the present invention. The components and specific ingredients are presented as being typical, and various modifications can be derived in view of the foregoing disclosure within the scope of the invention. All percentages, ratios and proportions herein are by weight, unless otherwise specified. All temperatures are in degrees Celsius unless otherwise specified.

### EXAMPLES

The starting MPSB is made according to following procedure: 400.0 grams of the nut form the shea butter tree are cracked into small pieces and placed into a one-liter vat of water. The water is then heated to 100° C. As the temperature increases, an oil phase develops on the surface of the water. The temperature is held for about 2 hours, after which the oil is decanted and passed through filter paper. The resulting butter is mild-processed shea butter according to the present invention. It is rich in unsaponifiable (from about 7% to about 15% by weight) and may be used in making the MPSB dimethicone copolyol derivatives, including MPSB DMC esters, of the present invention.

In the presence of 0.1% of dilauryl tin oxide, MPSB is esterified by adding the specified amount of the specified DMC (Examples 1-10). Under medium propeller agitation, the reaction mass is heated to, and held at, a temperature of from about 180° C. to about 200° C. for a period of about eight hours. During this time, the material clears and becomes homogeneous. After cooling, the products of Example 11-20 may be used without additional processing.

| | DMC | Grams |
|---|---|---|
| Example | Example | DMC |
| 11 | 1 | 458 |
| 12 | 2 | 712 |
| 13 | 3 | 1200 |
| 14 | 4 | 580 |
| 15 | 5 | 1000 |
| 16 | 6 | 1200 |
| 17 | 7 | 350 |
| 18 | 8 | 1205 |
| 19 | 9 | 1500 |
| 20 | 10 | 1470 |

While the illustrative embodiments of the invention have been described with particularity, it will be understood that various other modifications will be apparent to and can be readily made by those skilled in the art without departing from the spirit and scope of the invention. Accordingly, it is not intended that the scope of the claims appended hereto be limited to the examples and descriptions set forth hereinabove but rather that the claims be construed as encompassing all the features of patentable novelty which reside in the present invention, including all features which would be treated as equivalents thereof by those skilled in the art to which the invention pertains.

1. A dimethicone copolyol of shea butter conforming to the following structure: 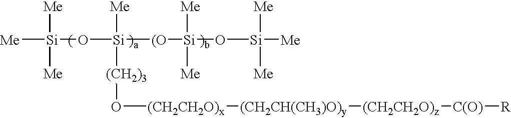
wherein
(i) Me is methyl;
(ii) a is an integer ranging from 1 to 20;
(iii) b is an integer ranging from 0 to 200;
(iv) x, y and z are independently integers ranging from 0 to 20; and
(v) R is derived from shea butter and comprises
from about 0.5% to about 2.0% by weight C₁₁H₂₃;
from about 0.5% to about 2.0% by weight C₁₃H₂₇;
from about 2.0% to about 6.0% by weight C₁₅H₃₁;
from about 25% to about 50% by weight C₁₇H₃₅;
from about 40.0% to about 60.0% by weight C₁₇H₃₃.

2. A dimethicone copolyol of shea butter of claim 1 wherein x is 0, y is 0 and z is 5.

3. A dimethicone copolyol of shea butter of claim 1 wherein x is 20, y is 20 and z is 20.

4. A dimethicone copolyol of shea butter of claim 1 wherein x is 0, y is 10 and z is 0.

5. A dimethicone copolyol of shea butter of claim 1 wherein x is 10, y is 10 and z is 10.

6. A dimethicone copolyol of shea butter of claim 1 wherein x is 20, y is 5 and z is 20.

7. A dimethicone copolyol of shea butter of claim 1 wherein x is 5, y is 10 and z is 20.

8. A dimethicone copolyol of shea butter of claim 1 wherein a is an integer ranging from 1 to 4.

9. A dimethicone copolyol of shea butter of claim 1 wherein b is 0.

10. A dimethicone copolyol of shea butter made by a transesterification reaction that conforms to the structure: 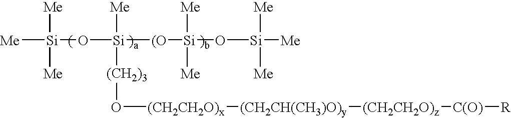
(i) Me is methyl;
(ii) a is an integer ranging from 1 to 20;
(iii) b is an integer ranging from 0 to 200;
(iv) x, y and z are independently integers ranging from 0 to 20; and
(v) R is derived from shea butter and comprises
from about 0.5% to about 2.0% by weight C₁₁H₂₃;
from about 0.5% to about 2.0% by weight C₁₃H₂₇;
from about 2.0% to about 6.0% by weight C₁₅H₃₁;
from about 25% to about 50% by weight C₁₇H₃₅;
from about 40.0% to about 60.0% by weight C₁₇H₃₃.

11. A dimethicone copolyol of claim 10 wherein the transesterification is conducted at a temperature of between 150° C. and 220° C. in the presence of a catalyst selected from the group consisting of methane sulfonic acid, tin compounds and titinate compounds.

12. A dimethicone copolyol of claim 11 made by a transesterification in the presence of a tin catalyst.

13. A dimethicone copolyol of claim 12 made by a transesterification where the tin catalyst is dilauryl tin oxide.

14. A dimethicone copolyol of mild-processed shea butter of claim 11 wherein x is 0, y is 0 and z is 5.

15. A dimethicone copolyol of mild-processed shea butter of claim 11 wherein x is 20, y is 20 and z is 20.

16. A dimethicone copolyol of mild-processed shea butter of claim 11 wherein x is 0, y is 10 and z is 0.

17. A dimethicone copolyol of mild-processed shea butter of claim 11 wherein x is 10, y is 10 and z is 10.

18. A dimethicone copolyol of mild-processed shea butter of claim 11 wherein x is 20, y is 5 and z is 20.

19. A dimethicone copolyol of mild-processed shea butter of claim 11 wherein x is 5, y is 10 and z is 20.

20. A dimethicone copolyol of mild-processed shea butter of claim 1 wherein a is an integer ranging from 1 to 4.

21. A dimethicone copolyol of mild-processed shea butter of claim 1 wherein b is 0.

22. A process for delivering antioxidants to the hair and skin by applying thereto a finished topical product comprising an effective concentration of a shea butter dimethicone copolyol that is made by a transesterification reaction and conforms to the structure: 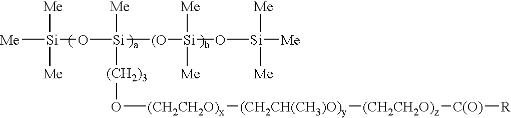
(i) Me is methyl;
(ii) a is an integer ranging from 1 to 20;
(iii) b is an integer ranging from 0 to 200;
(iv) x, y and z are independently integers ranging from 0 to 20; and
(v) R is derived from shea butter and comprises
from about 0.5% to about 2.0% by weight C₁₁H₂₃;
from about 0.5% to about 2.0% by weight C₁₃H₂₇;
from about 2.0% to about 6.0% by weight C₁₅H₃₁;
from about 25% to about 50% by weight C₁₇H₃₅;
from about 40.0% to about 60.0% by weight C₁₇H₃₃.

23. A process of claim 22 wherein x is 0, y is 0 and z is 5.

24. A process of claim 22 wherein x is 20, y is 20 and z is 20.

25. A process of claim 22 wherein x is 0, y is 10 and z is 0.

26. A process of claim 22 wherein x is 10, y is 10 and z is 10.

27. A process of claim 22 wherein x is 20, y is 5 and z is 20.

28. A process of claim 22 wherein x is 5, y is 10 and z is 20.

29. A process of claim 22 wherein a is an integer ranging from 1 to 4.

30. A process of claim 22 wherein b is 0.

31. A dimethicone copolyol of claim 1 where the shea butter is mild-processed.

32. A process of claim 22 where the shea butter is mild-processed.
